(19) United States
(12) Patent Application Publication
(10) Pub. No.: US 2025/0255669 A1
(43) Pub. Date: Aug. 14, 2025

(54) Surgical Navigation Using an AR Headset

(71) Applicant: Novarad Corporation, Provo, UT (US)

(72) Inventors: Wendell Arlen Gibby, Mapleton, UT (US); Steven Todd Cvetko, Draper, UT (US); Ryan West, Payson, UT (US)

(21) Appl. No.: 19/007,279

(22) Filed: Dec. 31, 2024

Related U.S. Application Data
(60) Provisional application No. 63/552,177, filed on Feb. 11, 2024.

(51) Int. Cl. A61B 34/00; A61B 34/20; A61B 90/00

(52) U.S. Cl. CPC A61B 34/00; A61B 2034/2057; A61B 2090/365

(57) ABSTRACT

Technology is described that includes a method of surgical navigation. The method can include aligning an image data set with a body of a person. The image data set may include a virtual surgical annotation with visual attributes identifying structure to be treated. Locations of a portion of an instrument may be tracked with respect to the virtual surgical annotation (e.g., drill bit tips, burrs, scalpels, etc.). The visual attributes of at least one sub-area of the virtual surgical annotation may be modified based in part on tracked locations of the portion of the instrument, using an AR headset. For example, the sub-areas maybe changed from red to green, black or transparent as seen through the AR headset.

### PRIORITY DATA

This application claims the benefit of U.S. Provisional Patent Application Ser. No. 63/552,177, filed Feb. 11, 2024, which is incorporated herein by reference.

### BACKGROUND

Mixed or augmented reality is an area of computing technology where views from the physical world and images from virtual computing worlds may be combined into a mixed reality world. In mixed reality, people, places, and objects from the physical world and virtual worlds become a blended visual and audio environment. A mixed reality experience may be provided through existing commercial or custom software along with the use of VR (virtual reality) or AR (augmented reality) headsets.

Augmented reality (AR) is an example of mixed reality where a live direct view (or an indirect view) of a physical, real-world environment is augmented or supplemented by computer-generated sensory input such as sound, video, graphics or other data. Augmentation is performed as a real-world location is viewed and in context with environmental elements. With the help of advanced AR technology (e.g. adding computer vision and object recognition) the information about the surrounding real world of the user becomes interactive and may be digitally modified.

### BRIEF DESCRIPTION OF THE DRAWINGS

FIG. 1 is an illustration of an example system and a method for surgical navigation.

FIG. 2 is an illustration of an example a virtual surgical annotation that may be used on the leg of patient.

FIG. 3 illustrates an example of the use of breakout views with a virtual surgical annotation on a cadaver leg.

FIG. 4 is an illustration of an example of a view of a leg with an immersive view and three breakout views.

FIG. 5 is similar to FIG. 4 but depicts a later point in time during the use of an instrument.

FIG. 6 is a flowchart illustrating an example method of surgical navigation.

FIG. 7 is a flow chart illustrating a second example method of surgical navigation for a medical procedure using an AR headset.

### DETAILED DESCRIPTION

Reference will now be made to the examples illustrated in the drawings, and specific language will be used herein to describe the same. It will nevertheless be understood that no limitation of the scope of the technology is thereby intended. Alterations and further modifications of the features illustrated herein, and additional applications of the examples as illustrated herein, which would occur to one skilled in the relevant art and having possession of this disclosure, are to be considered within the scope of the description.

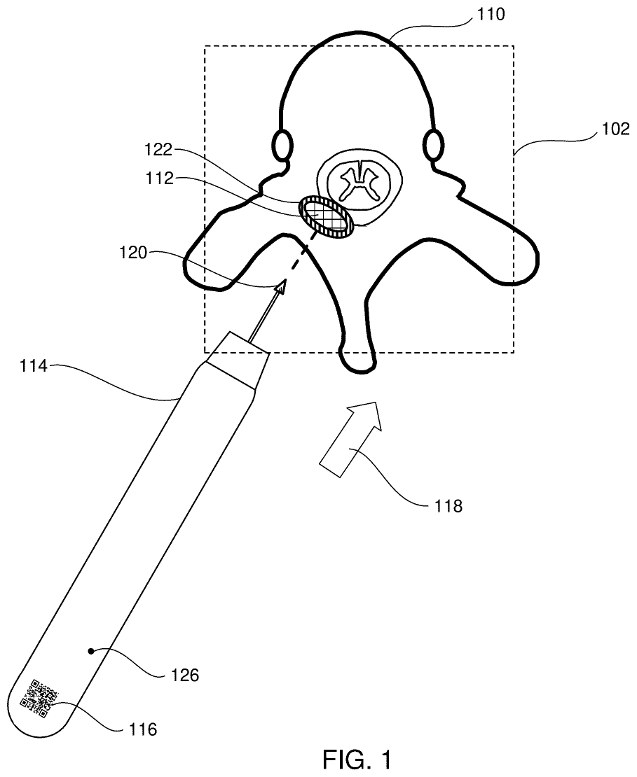

FIG. 1 is an illustration of a system and/or a method for surgical navigation. An image data set 102 may be aligned with a body of a person. The image data set may be 3D (three dimensional) medical imaging, such as a CT scan or an MRI scan, and the image data set may include bones 110 (e.g., a vertebrae), and other hard tissue and/or soft tissue that can be captured using known medical imaging methods.

The image data set 102 may include a virtual surgical annotation 112 with visual attributes identifying structure to be treated. The visual attributes may be attributes applied to voxels or pixels of the image data set such as color, intensity, contrast, opacity, etc. The virtual surgical annotation may also be applied separately from the image data set 102 (e.g., using hand alignment).

The locations of a portion 120 of an instrument 114 may be tracked with respect to the virtual surgical annotation 112 using the AR headset. The portion 120 of the instrument being tracked may be: a rotary die grinder bit, a drill bit, a burr, a scalpel, an electrocautery tip, a cryosurgical probe, a saw blade or another tool that can remove and/or cut the hard or soft tissue of a patient. The instrument 114 may be a medical instrument identified and tracked by using the outline of the instrument. Alternatively, the instrument may be identified and tracked using an optical code 116 or a visible marker 126.

The visual attributes of at least one sub-area of the virtual surgical annotation 112 may be modified. This modification may occur based in part on tracked locations of the portion of the instrument (e.g., a drill bit tip or burr), using the AR headset. The visual attributes that were modified for one or more sub-areas (e.g., voxels or pixels) may represent anatomical structure that has been removed from the patient or modified in the patient by the portion of the instrument. The modifying of the visual attributes may include modifying the visual attributes of one or more sub-areas of the virtual surgical annotation by changing: color, intensity, opacity, pattern, animations or another visual indicator at the sub-area locations. A sub-area may be one or more voxels, pixels or other picture elements that make up the virtual surgical annotation 112 in 3D. The sub-areas may have their color changed, for example, from red to black when the portion of the instrument or the tip of a drill or rotary cutting tool have been tracked as being at that location or 3D coordinate in the virtual surgical annotation 112.

Guidance may also be provided to the user or medical professional using the AR headset. For example, the guidance may tell the user which direction to move the instrument or medical instrument. The guidance may tell the user to move the instrument in a specific relative direction (e.g., left or right) or absolute direction (e.g., up or down) so that the virtual surgical annotation may be traversed in every direction by the tip of the instrument as controlled by the medical professional. The guidance may be visual directional guidance (e.g., an arrow 118) or audible directional guidance for movement of the medical instrument by a user with respect to the virtual surgical annotation 112.

An image data set can be aligned to the body of the person using a marker or other alignment systems. Medical imaging may be obtained and aligned with a body of a person. For example, a CT (computed tomography) scan, MRI (magnetic resonance imaging) image or other imaging may be overlaid on the patient and used as a reference for aspects of a patient's anatomical structure being operated on. U.S. Pat. Nos. 9,892,564; 10,475,244; 11,004,271;10,010,379; 10,945,807; 11,266,480; 10,825,563; 11,237,627; 11,287,874; U.S. patent application Ser. Number: 17/706,462 entitled “Using Optical Codes with Augmented Reality Displays”; and U.S. patent application Ser. Number: 17/536,009 entitled “Image Data Set Alignment for an AR Headset Using Anatomic Structures and Data Fitting”; and U.S. patent application Ser. Number: 17/978,962 entitled “3D Spatial Mapping in a 3D Coordinate System of an AR Headset Using 2D Images” describe methods and systems for aligning an image data set from medical imaging devices with a body of a person and these descriptions are incorporated in their entirety by reference herein. An image data set may be aligned to the body of the person using: markers, optical codes, radiopaque markers, 2D imaging, morphometrics or other systems for alignment of 3D image data sets. These patents also describe a wide variety of medical imaging types that may be used to obtain 3D image data sets.

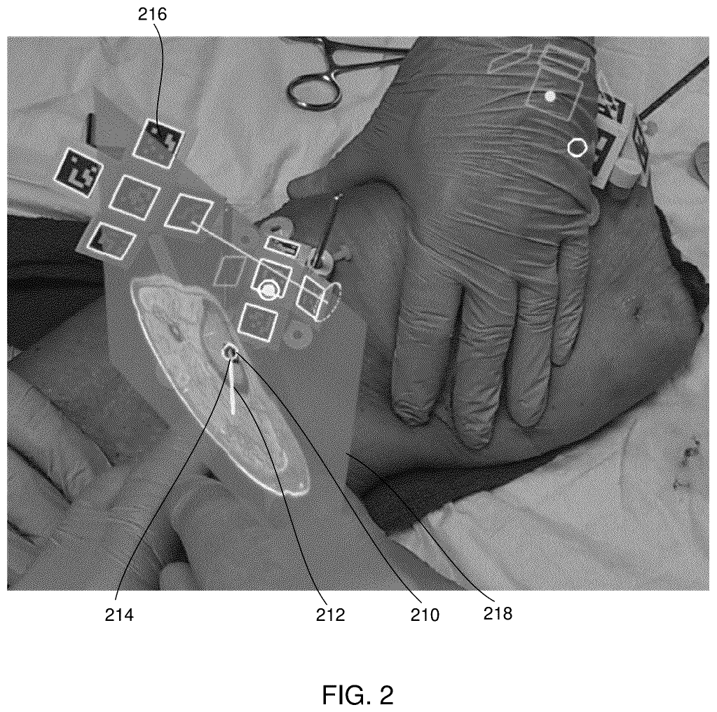

FIG. 2 illustrates that a virtual surgical annotation 210 may be used on the leg of a patient (cadaver leg shown in illustration). For example, there may be an area of bone that a medical professional is going to remove. In another example, a tumor may need to be removed or an impingement of the femur on to the acetabulum (socket) of the hip may need to be ground down. The virtual surgical annotation 210 or virtual surgical plan may be seen through the AR headset.

The AR headset can also track an instrument that the medical professional may use to remove or cut out the area identified by the virtual surgical annotation 210. In FIG. 2, the instrument or medical instrument is hidden behind a slice view 218 of the image data set, but a virtual representation of the instrument 212 is depicted. In addition, a virtual representation of a tip 214 of the medical instrument is illustrated. The medical instrument may be rotary die grinder (e.g., a Dremel® style tool), a burring tool, drilling tool, ultrasonic bone scalpel, or another tool for removing tissue, etc.

Then the AR headset can track where a physical tip of a medical instrument has been moved in the 3D coordinate space of the AR headset. To enable the medical professional to visibly track where the tip of the instrument has been, the color of voxels (or pixels or pels (picture elements)) in the virtual surgical annotation 210 can be changed from a first color (e.g., red or white) to a second color (e.g., green or black) at each location the tip of the instrument has been located at or visited. By analogy, it may appear to the medical professional as though they are erasing the virtual surgical annotation 210 (e.g., the pre-planned surgical area) because the AR headset is tracking where the tip of the instrument or medical tool has been and a change is made visually in the virtual surgical annotation 210 to represent any sub-area the tip has touched.

The image data set may have also been aligned to the body of the person or patient using the optical codes 216 on the patient. Other alignment methods for aligning the image data set with the body of the person can also be used as described earlier.

In another similar example, the virtual surgical annotation 210 may be a colorized area that has been marked by the medical professional. As the medical professional moves the tip of the medical instrument through the 3D space defined by the virtual surgical annotation 210, the sub-volumes (e.g., voxels or pixels, etc.) in the virtual surgical annotation 210 may change have their color changed by the AR headset, so that the medical professional can visually see that the unwanted bone and/or tissue of the patient's body have removed (e.g., tumor removal).

Additionally, various zones in the virtual surgical annotation 210 may have different colors, textures or animations. For example, a red zone may represent the tumor to be removed, a yellow zone may represent a tumor margin, and a green zone may represent an area of patient anatomy that should not be removed.

A feature to automatically create a margin zone for each virtual surgical annotation 210 may be provided. A medical professional may enter a value into a setting in the software for a tolerance (e.g., 0.25 cm) around a virtual surgical annotation 210 that is used to automatically generate a margin (e.g., a 3D margin) around the virtual surgical annotation 210.

A warning notification may also be generated when a medical professional or user moves the tip of an instrument outside of a boundary of the virtual surgical annotation or outside a zone of the virtual surgical annotation. For example, a visual warning may flash in the AR headset by turning the virtual tool and virtual tip red when the medical profession moves outside a zone or outside the virtual surgical annotation. Alternatively, red text or another graphical warning may be displayed to warn the medical professional about going outside the virtual surgical annotation or a zone of the virtual surgical annotation.

In another example, an audible warning that is a tone, beep or message may be played as the instrument tip moves outside the virtual surgical annotation or a zone of the virtual surgical annotation. Different tones may be played when certain zones are exited. For example, leaving a central zone to enter a margin zone may play a pleasant tone, while going outside a margin zone into tissue that should not be removed may play an unpleasant tone.

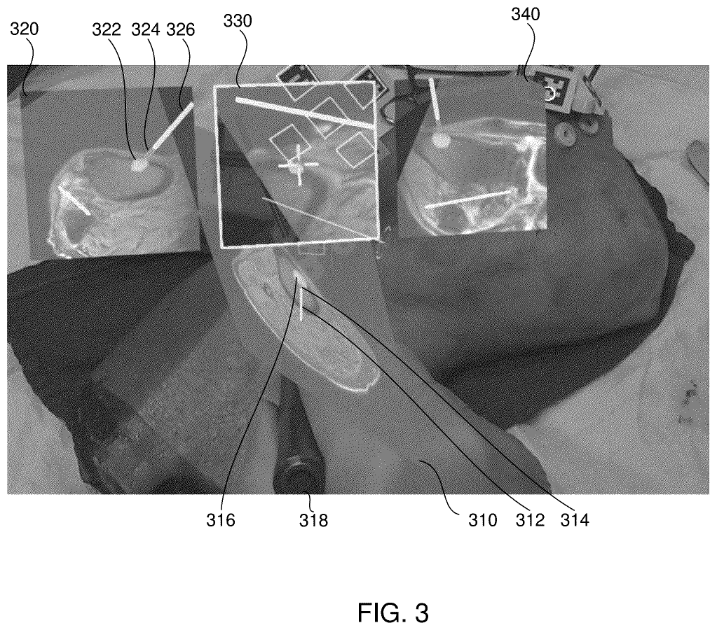

In the example in FIG. 3, the virtual representation of a tip 214 or burr of the medical instrument may be red for contrast purposes. In contrast, a section of the bone where the virtual surgical annotation 210 is located may be colored pre-operatively as green (e.g., setting an initial visual attribute) using the virtual surgical annotation 210. The medical professional may set or enter the diameter of the burr of the instrument and then start using the instrument to “virtually erase” and also physically resect and vacuum out the section of the bone that needs to be removed. By viewing the change in color in the virtual surgical annotation 210, the medical professional knows that the correct tissue area (e.g., bone) and/or margins have been removed. Similarly, if the medical professional uses the tool on a tumor and all of the green areas in the virtual surgical annotation 210 have been turned transparent, black or red, then the medical professional may know that the tumor and/or tumor margins have been hit by the tip of the tool and have been removed. Any type of medical instrument that can remove or cut anatomical structure can be used. For example, the medical professional may use a burr, a scalpel, ultrasonic bone scalpel, ultrasonic cutter or any other surgical resection type of tool.

FIG. 3 illustrates the use of breakout views with this technology, as applied on a cadaver leg. The breakout views may be considered virtual camera views of the image data set from perspectives other than the medical professional's physical view. In the bottom center is an immersive view, where an oblique slice 310 of an image data set is illustrated. A full image data set or other views may be displayed in bottom center location as determined by a medical professional. A virtual instrument 312 may be aligned with the real instrument 318 the medical professional is holding. The virtual tip 314 of the virtual instrument 312 may be displayed for the medical professional and may allow the medical professional to determine where the real cutting drill bit, burr or scalpel is located: in the 3D coordinate system, in the patient's body and with respect to the virtual surgical annotation 316. A side breakout view 320 is illustrated where the virtual instrument 326, virtual instrument tip 324, and virtual surgical annotation 322 are displayed with a view of the image data set. Two additional breakout views, including a top view 330 and an opposing side view 340 are also illustrated.

Referring again to FIG. 1, the virtual surgical annotation 112 may have two or more zones 122, 112 as part of the virtual surgical annotation 112. More specifically, a second zone may be created as a margin zone 122. For example, a medical professional may mark a tumor and then provide a certain parameter that represents an expansion distance for the margins of the tumor. For example, this parameter may be 0.25 cm in 3D surrounding the virtual surgical annotation 112 defined by the medical professional. Accordingly, the margin zone 122 may be added to and/or become part of the virtual surgical annotation 112. This margin zone 122 may also be represented as the second outer zone. In another example, the medical professional may mark the tumor with a virtual surgical annotation 112 and a setting may already be stored for automatically creating a margin of a certain distance from the anatomical structure. For instance, the margin may be set to automatically extend 0.5 cm past the area marked for a tumor or any other anatomical structure being operated on.

The zones may be set to have different colors, patterns or numbers for each zone. For example, there may be a central red zone, a surrounding yellow zone, and then a green zone or vice versa. The colors of the zones may represent the margin of a tumor, danger zones or other aspects of the anatomic structure that a medical professional wants to denote or reference during a medical procedure. The zones do not need to be oval as illustrated in FIG. 1 but the zones may be any desired shape, including: rectangular, square, box shaped, spherical, oblong, any 2D shape, any regular 3D shape, or any irregular 3D shape.

The software in the AR headset may provide audible feedback or visual feedback to a medical professional. The visual feedback may be arrows 118, colors, animations or other visual indicators show a direction in which the medical professional may move the instrument in order to ensure that a tip of an instrument may travel to every part of the virtual surgical annotation 112. The visual indicators may also provide instructions that are part of a surgical guide to instruct the medical professional on a path to take to reach the virtual surgical annotation 112.

Any audible directions that are used may instruct the medical professional to “move right”, “move left”, “move up slowly”, etc. within a virtual surgical annotation 112. The user may also receive visual or audible feedback when the resection or surgical removal of part of an organ or anatomic structure is complete. For example, if every sub-location of the virtual surgical annotation 112 has been visited by the tip of the medical instrument, then the system will know that surgical removal is complete. Such visual or audible instructions can be provided in the immersive view of the AR headset or in the breakout 3D navigation views.

This technology may track and/or manage: the location of the body of the patient, the alignment of the image data set with the body, the location of the instrument, the location of a portion of the instrument that removes or cuts tissue, and the alignment of a virtual instrument and the tip of the virtual instrument with the physical instrument. These items may be tracked in 3D coordinate space of the AR headset. The AR headset allows the medical professional to map or identify the pathology using a 3D shape or volume defined as the virtual surgical annotation, and the image data set can be co-registered to the body of the patient or person. This alignment and tracking allows the medical personnel (e.g., doctor) to use the non-invasive surgical devices and/or the instrument and sculpt away anatomic material while providing visual indictors to the medical personnel to assist with staying within the boundaries and accessing the entire volume of the virtual surgical annotation. Changing the visible attributes of the sub-areas of the virtual surgical annotation provides a high degree of confidence that the correct total area(s) of the anatomic structure will be treated as represented visually.

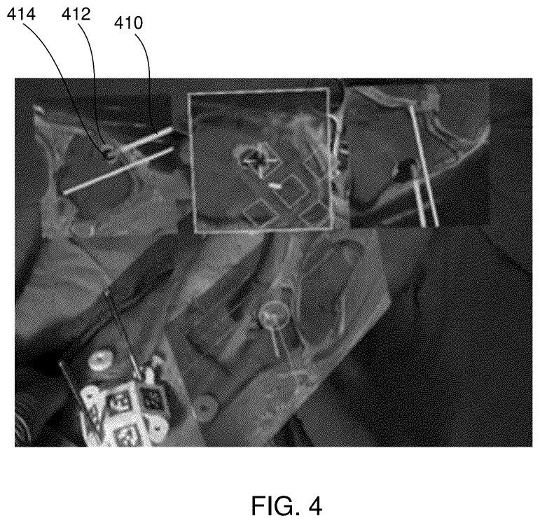

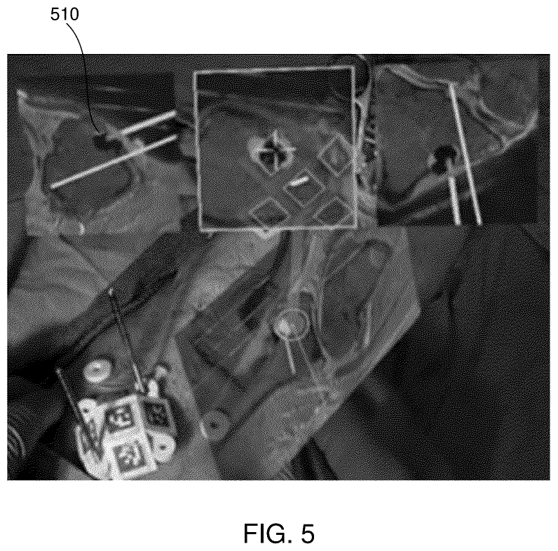

FIG. 4 illustrates a view of a leg with an immersive view and three breakout views. The physical instrument, as represented by the virtual instrument 410, may be used to sculpt away some of the tissue 412 (that may be marked green) within the virtual surgical annotation 414. FIG. 5 illustrates the same view of the leg and virtual surgical annotation but at a later point in time. Some of the tissue 510 (e.g., marked green) in FIG. 5 has been removed or sculpted away within the virtual surgical annotation and the removed sub-areas or portions of the virtual surgical annotation have been set to black as the tip of instrument has traveled through the sub-areas.

In this technology, the virtual surgical annotation is not just displayed to a user or medical professional but the virtual surgical annotation becomes an active part of guiding the instrument used by the medical professional or user in the 3D coordinate space. In addition, the instrument or medical instrument may act as a 3D “eraser” by erasing portions of the patient's anatomic structure that a medical professional has selected for removal. The removal of the anatomic structure identified by the virtual surgical annotation is also illustrated graphically in the AR headset as the virtual surgical annotation changes in the sub-areas where a cutting tip of the medical instrument travels. In a sense, the medical professional has an “eraser” at the tip of a high speed drill or burr and the AR headset can produce the visualization of the virtual surgical annotation that represents the “erasing” operation.

The ability of a sub-portion of the virtual surgical annotation to immediately change as a medical instrument moves around in a patient's tumor and is resected can provide an immediate visual verification about the anatomic structure the medical professional has resected or has not resected. The processes executing in the AR headset are configured to let the medical professional know where the tip of the medical instrument has traveled and how much of the anatomic structure associated with the virtual surgical annotation has been removed and has not been removed. Without an electronic guide, knowing where the tip of the medical instrument is inside of opaque tissue (bone, under muscle, etc.) and knowing the amount of tissue already removed is difficult for medical personnel to determine.

The desire to remove anatomic structure or bodily tissues is common in orthopedic operations, as mentioned already. For example, a person may have a bit of bony overgrowth on their femur. The bony growth may bump into the person's acetabulum in the joint of the hip, which may rub and cause pain. When the medical professional performs a medical procedure to resect the undesirable growth or tissue, then the medical professional may want to trim away some of the bone. However, the medical professional may not know much bone to resect or may not have an exact knowledge of what needs to be removed. The medical professional can estimate the bone to be removed off of 2D images (e.g., 2D x-ray generated images) in the operating room but using a 2D image in the operating room can lead to inaccuracies in the resection. The medical professional is not likely to be able to take a new or repeated CT scan or MRI in the operating room, and this is due to size and expense of such equipment, which generally precludes such equipment from being used in an operating room. Generally, CT scanners are not available in an operating room, but if a CT scanner is available in an operating room, then the image quality of the CT scan is generally poor. Accordingly, this technology enables resection of a bony overgrowth on a femur with a high degree of precision using the virtual surgical annotation and processes and described earlier.

In another example, a medical professional may want to remove 1 cm of bone from deep within a hip. The location of the bone to be removed may not be exposed but may be accessed using minimally invasive procedures with a scope. The medical professional does not want to just start drilling without confidence that the right amount of bone in the right location can be removed. A CT scan can be captured prior to the medical operation or surgery. The medical professional can then make a plan on exactly how much bone is to be removed and the volume can be marked with a virtual surgical annotation. Both the CT scan and the virtual surgical annotation can be aligned with the correct anatomical structure of the body of the person. The instrument position can be tracked by the AR headset (e.g., using a tracker or the shape of the instrument). The portion of the instrument that performs tissue removal or the instrument tip (e.g., a drill bit or burr) can also be tracked. Even if the drill bit or burr cannot be seen by the medical professional because the instrument tip is within the anatomical structure of the patient, the system can still track the instrument tip using the portion of the instrument that is visible to the AR headset. Accordingly, the medical professional can virtually watch the removal of the bone, even though the instrument tip is inside the bone where the medical professional cannot see the instrument tip in the bone. In other words, the medical professional will see the location of the drill bit or burr on the AR headset even if the drill bit or burr is not directly visible. More specifically, the medical professional will be able to see the locations or sub-areas of the virtual surgical annotation change color when the instrument tip is calculated as being at that position of the virtual surgical annotation. The medical professional may also receive extra guidance regarding where to move the tip of the instrument in order to remove or cut tissue, as discussed earlier. For example, the AR headset may provide graphical, audible or tactile instructions (e.g., haptic feedback) to move up, move down, move left, move right, move forward, move back, etc.

This technology provides guidance to a medical professional using a tracking system that can track a physical instrument and display a virtual instrument aligned with the physical instrument, as the physical instrument (particularly the tip of the instrument) moves through the image data set and the virtual surgical annotation displayed in virtual space. In addition, the virtual space of the image data set (e.g., CT scan or MRI scan) can be aligned to the physical space. Therefore, as the medical professional removes the virtual representation of bone or other tissue in the virtual surgical annotation, the medical professional will have successfully removed the real bone that is to be resected. By analogy, it is like the medical professional has a 3D eraser that can be used in surgery.

The present technology may be applied in a number of medical applications. One example medical application that has already been described may be femur acetabular impingement. A medical professional can use this technology to sculpt away a bone growth that creates the impingement. In this type of medical procedure, it may be difficult or not possible to see the bone during the procedure and the medical professional may not know how much bone needs to be removed. Using the AR headset to display the virtual surgical annotation(s) can help to avoid the problems that may occur from not being able to directly see the bone that needs to be removed and not knowing how much bone needs to be removed.

The removal of a spinal tumor or other tumors is another area where the present technology may be used to improve patient outcomes. In this type of procedure, the medical professional or doctor may need to resect bone, and the medical professional would also like to resect the bony margins of the tumor. In surgery, it can be difficult to find the margins of a spinal tumor or sometimes even difficult to determine what tissue belongs to the tumor and what tissue does not belong to the tumor due to bleeding, seepage and material being removed during a medical procedure. However, where the tumor's 3D volume has been marked out virtually in advance using the virtual surgical annotation, then as the burr, drill or saw is moved within the virtual surgical annotation and the medical professional unroofs the tumor then the medical professional may clearly identify the tumor margins. Further, as the color of the virtual surgical annotations and/or zones change, then the medical professional knows the right margin for the tumor has been resected.

This technology may also apply to the use of a cryosurgical probe that may be used to freeze a tumor. The diameter of tissue that is expected to be frozen as the cryosurgical probe is moved near or within the patient tissues is known in advance. As the cryosurgical probe moves, then an area of the appropriate diameter in the virtual surgical annotation may have the color or pattern changed based on the known freezing diameter of the cryosurgical probe. As the color changes within the virtual surgical annotation, then the medical professional can see that the appropriate area of the tumor has been frozen or treated.

MRI scans or image can be quite useful in the medical field because the soft tissue can be seen clearly. If a patient has a sarcoma in their leg, then a mass may be visible the MRI image. However, it may be difficult during a medical procedure to remove the sarcoma and to determine where the margins of the sarcoma are. In this case, the margins of the sarcoma can be colorized in the virtual surgical annotation. Then as the medical professional moves the wand, drill bit, burr, scalpel, ultrasonic bone scalpel, cutting device, or cautery device, the AR headset can record where the doctor has moved the instrument and instrument tip and know that the correct margins have been removed or treated.

Minimally invasive surgery has also become more widespread and can be improved by this technology. Because it is better not to surgically open up a patient's back or other internal areas due to complications and/or recovery pain issues, many medical procedures are being performed through a small scope. While a doctor can install pedicle screws percutaneously, this is not feasible when removing the bone, lamina, and/or decompressing the spine. It can also be challenging for a doctor to see where their instrument is located in the patient during such minimally invasive medical procedures. Minimally invasive surgery can be challenging because the doctor is looking through a tiny hole with a scope 2 mm in diameter. Using the virtual surgical annotation and visual attribute modification process, the bone can be marked, and as the medical professional cuts the bone away using an instrument tip, then the color or pattern of the sub-areas of the virtual surgical annotation may be changed. The results is that the medical professional knows which sub-areas have been treated or removed.

In this technology, doctors or medical professionals can use an instrument and instrument tip as a virtual wand. In this case, the virtual wand may be a high speed drill with a burr on the end that is 3 mm (millimeters) in diameter and spinning at 75,000 RPMs (rotations per minute). Then as the doctor moves the burr of the instrument through all of the volume of the virtual surgical annotation, then the medical professional knows the patient's tissue (e.g., bone) is gone in that area. The instrument may be tracked in 3D and doctor's goal may be to move through and match the entirety of 3D surgical annotation volume. The doctor may create a virtual surgical annotation (e.g., in a color or pattern) and then change it or “erasing it” by changing it to black, green, clear or another background color as the instrument tip passes through sub-areas.

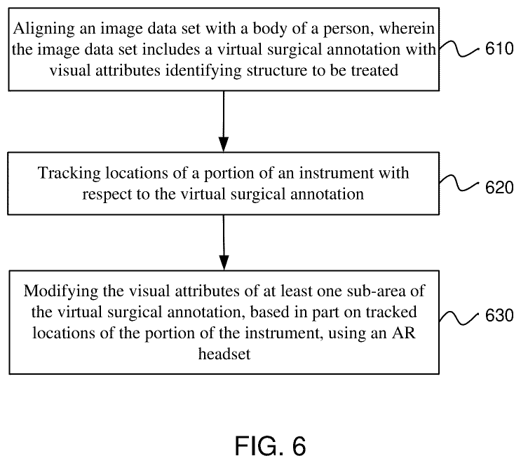

FIG. 6 illustrates a method of surgical navigation. The method may include aligning an image data set with a body of a person or patient, as in block 610. The image data set may include a virtual surgical annotation with visual attributes identifying structure to be treated. The virtual surgical annotation may be a 3D (three dimensional) volume in any shape. For example, the shape may be defined to match any shape of an irregular tumor or a space in tissue (e.g., bone) to be opened up with a burr.

Another operation may be tracking locations of a portion of an instrument with respect to the virtual surgical annotation, as in block 620. The portion of the instrument being tracked may be a tip of an instrument, such as a rotary die grinder bit, a drill bit, a burr, a scalpel, an electrocautery tip, a cryosurgical probe, or a saw blade. While the entire instrument may be tracked in order to track the bit of the instrument, the bit or blade is useful to track because that is the location where tissue in the patient may be removed, ground down or otherwise treated.

The visual attributes of at least one sub-area of the virtual surgical annotation may be modified, based in part on tracked locations of the portion of the instrument, as in block 630. These operations may also be performed using software loaded on an AR headset. The visual attributes that are modified may represent anatomical structure that has been removed or modified by the portion of the instrument. For example, modifying the visual attributes may include modifying the visual attributes of a sub-area of the virtual surgical annotation by changing the color, intensity, opacity or pattern of a sub-area. The visual attributes may be modified from a first set of visual attributes (e.g., red) to second set of visual attributes (e.g., black). Visual directional guidance or audible directional guidance may be provided for movement of the instrument by a user with respect to the virtual surgical annotation.

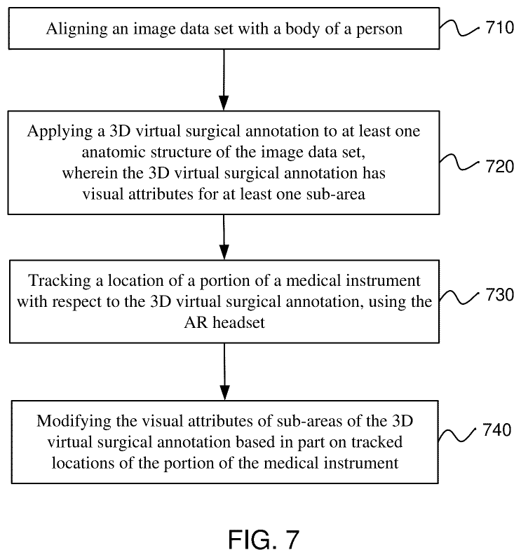

FIG. 7 illustrates a method of surgical navigation for a medical procedure using an AR headset. The method may include aligning an image data set with a body of a person, as in block 710. Another operation may be applying a 3D virtual surgical annotation to at least one anatomic structure of the image data set, as in block 720. The 3D virtual surgical annotation may have visual attributes for at least one sub-area of the 3D virtual surgical annotation. The 3D virtual surgical annotation may be a 3D (three dimensional) volume in any shape set by a medical professional.

A location of a portion of a medical instrument may be tracked with respect to the 3D virtual surgical annotation, using the AR headset, as in block 730. In addition, a graphical virtual medical instrument may be displayed in alignment with the physical medical instrument that is visible through the lenses (e.g., waveguides) of the AR headset. Examples of the medical instrument may be: a rotary die grinder, a drill, a burr, a scalpel, an electrocautery tool, a cryosurgical tool, ultrasonic bone scalpel, or a saw. The portion of the medical instrument that may be tracked can include: rotary die grinder bit, a drill bit, a burr, a scalpel, a cryosurgical probe, an electrocautery blade, or other tissue removal or cutting tips.

The visual attributes of sub-areas of the 3D virtual surgical annotation may be modified based in part on tracked locations of the portion of the medical instrument, as in block 740. The visual attributes that were modified may represent that anatomical structure has been modified or removed by the medical instrument at the tracked locations, as displayed using the AR headset. The visual attributes of a sub-area may be modified by changing at least one of: a color attribute, an intensity attribute, an opacity attribute or a pattern. Accordingly, modifying the visual attributes may be changing a color from red to green or black, or changing the opacity of voxels or pixels of the 3D virtual surgical annotation to be transparent.

The portion of a virtual instrument may be displayed as being overlaid on an anatomical structure to allow visualizing a tip of the medical instrument hidden within a pattern or anatomical structure visible. The system may also provide visual or audible directional guidance for movement of the medical instrument within the 3D virtual surgical annotation by a user.

This technology may also be described as erasing the sub-areas of the 3D virtual surgical annotation by setting the visual attributes of the sub-areas of the 3D virtual surgical annotation to another color or to be a background color based in part on tracked locations of the portion (e.g., the tip) of the medical instrument. In this configuration, the term erasing may mean setting the color of the sub-areas to another color or a background color of the image data set. In a further configuration, the color may be removed and the locations of the tip of a medical instrument within the 3D virtual surgical annotation can remove that portion of the virtual shape so that the sub-area is transparent or see-through in the AR headset. The visual attributes that were erased may represent anatomical structure that has been removed or modified (e.g., cut) by the medical instrument at the tracked locations of the portion (e.g., tip) of the medical instrument. For example, sub-areas of the 3D virtual surgical annotation may be set to transparent or another color (e.g., black, white or a background color of the image data set).

Some of the functional units described in this specification have been labeled as modules, in order to more particularly emphasize their instrumentation independence. For example, a module may be instrumented as a hardware circuit comprising custom VLSI circuits or gate arrays, off-the-shelf semiconductors such as logic chips, transistors, or other discrete components. A module may also be instrumented in programmable hardware devices such as field programmable gate arrays, programmable array logic, programmable logic devices or the like.

Modules may also be instrumented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices. The modules may be passive or active, including agents operable to perform desired functions.

The technology described here can also be stored on a computer readable storage medium that includes volatile and non-volatile, removable and non-removable media instrumented with any technology for the storage of information such as computer readable instructions, data structures, program modules, or other data. Computer readable storage media include, but is not limited to, RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which can be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared, and other wireless media. The term computer readable media as used herein includes communication media.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology can be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of instrumenting the claims. Numerous modifications and alternative arrangements can be devised without departing from the spirit and scope of the described technology.

What is claimed is:

1. A method of surgical navigation, comprising:
aligning an image data set with a body of a person, wherein the image data set includes a virtual surgical annotation with visual attributes identifying structure to be treated;
tracking locations of a portion of an instrument with respect to the virtual surgical annotation; and
modifying the visual attributes of at least one sub-area of the virtual surgical annotation, based in part on tracked locations of the portion of the instrument, using an AR headset.

2. The method as in claim 1, wherein the visual attributes that are modified represent anatomical structure that has been removed or modified by the portion of the instrument.

3. The method as in claim 1, wherein modifying the visual attributes further comprises modifying the visual attributes of the at least one sub-area of the virtual surgical annotation by changing at least one of: color, intensity, opacity or pattern.

4. The method as in claim 1, wherein the virtual surgical annotation is a 3D (three dimensional) volume.

5. The method as in claim 1, wherein the portion of the instrument is at least one of: a rotary die grinder bit, a drill bit, a burr, a scalpel, an electrocautery tip, a cryosurgical probe, or a saw.

6. The method as in claim 1, further comprising providing visual directional guidance or audible directional guidance for movement of the instrument by a user with respect to the virtual surgical annotation.

7. A method of surgical navigation for a medical procedure using an AR headset, comprising:
aligning an image data set with a body of a person;
applying a 3D virtual surgical annotation to at least one anatomic structure of the image data set, wherein the 3D virtual surgical annotation has visual attributes for at least one sub-area of the 3D virtual surgical annotation;
tracking a location of a portion of a medical instrument with respect to the 3D virtual surgical annotation, using the AR headset; and
modifying the visual attributes of sub-areas of the 3D virtual surgical annotation based in part on tracked locations of the portion of the medical instrument.

8. The method as in claim 7, wherein the visual attributes that were modified represent that anatomical structure has been modified or removed by the medical instrument at the tracked locations, using the AR headset.

9. The method as in claim 7, wherein modifying the visual attributes further comprises modifying the visual attributes of the at least one sub-area by changing at least one of: a color attribute, an intensity attribute, an opacity attribute or a pattern.

10. The method of claim 7, wherein modifying the visual attributes further comprises changing a color or opacity of voxels of the 3D virtual surgical annotation.

11. The method as in claim 7, wherein the 3D virtual surgical annotation is a 3D (three dimensional) volume.

12. The method as in claim 7, wherein the portion of the medical instrument is at least one of:
a rotary die grinder bit, a drill bit, a burr, a scalpel, an electrocautery tip, a cryosurgical probe, ultrasonic bone scalpel, or a saw blade.

13. The method as in claim 7, further comprising providing visual directional guidance or audible directional guidance for movement of the medical instrument by a user within the 3D virtual surgical annotation.

14. The method of claim 7, further comprising displaying a graphical virtual medical instrument aligned with the medical instrument that is visible.

15. The method of claim 7, further comprising displaying the portion of a virtual instrument overlaid on an anatomical structure to allow visualizing a tip of the medical instrument hidden within a pattern or anatomical structure visible.

16. The method of claim 7, wherein the medical instrument is a rotary die grinder, a drill, a burr, a scalpel, an electrocautery tool, a cryosurgical tool, or a saw.

17. The method of claim 7, further comprising providing a warning notification when the portion of the medical instrument passes outside of a boundary of the virtual surgical annotation.

18. A method of surgical navigation for a medical procedure using an AR headset, comprising:
aligning an image data set with a body of a person;
applying a 3D virtual surgical annotation to at least one anatomical structure of the image data set, wherein the 3D virtual surgical annotation has visual attributes for at least one sub-area of the 3D virtual surgical annotation;
tracking a location of a portion of a medical instrument with respect to the 3D virtual surgical annotation, using the AR headset; and
erasing the at least one sub-area of the 3D virtual surgical annotation by setting the visual attributes of the at least one sub-area of the 3D virtual surgical annotation to transparent or another color based in part on tracked locations of the portion of the medical instrument.

19. The method as in claim 18, further wherein the visual attributes that were erased represent anatomical structure has been removed or modified by the medical instrument at the tracked locations of the portion of the medical instrument, using the AR headset.

20. The method as in claim 18, wherein the portion of the medical instrument is a rotary die grinder bit, a drill bit, a burr, or a scalpel.

21. The method of claim 18, further comprising displaying the portion of a virtual instrument overlaid on an anatomical structure to allow visualizing a tip of the medical instrument hidden within the anatomical structure visible.

22. The method of claim 18, wherein setting sub-areas of the 3D virtual surgical annotation to transparent or another color further comprising setting the at least one sub-area to black, white or a background color of the image data set.
